(19) United States
(12) Patent Application Publication
(10) Pub. No.: US 2025/0369912 A1
(43) Pub. Date: Dec. 4, 2025

(54) SOLID RESERVOIR REFERENCE ELECTRODE

(71) Applicant: THE ROYAL INSTITUTION FOR THE ADVANCEMENT OF LEARNING/MCGILL UNIVERSITY, Montréal (CA)

(72) Inventors: Ngoc Anh Minh TRAN, Montréal (CA); Gabriele CAPILLI, Montréal (CA); Thomas SZKOPEK, Outremont (CA)

(21) Appl. No.: 18/877,306

(22) PCT Filed: Jul. 4, 2023
(86) PCT No.: PCT/CA2023/050906

§ 371 Date: Dec. 20, 2024

Related U.S. Application Data
(60) Provisional application No. 63/358,300, filed on Jul. 5, 2022.

(51) Int. Cl. G01N 27/30; A61B 5/266; G01N 27/327; G01N 27/333; G01N 27/414

(52) U.S. Cl. CPC G01N 27/301; A61B 5/266; G01N 27/302; G01N 27/327; G01N 27/333; G01N 27/4145

(57) ABSTRACT

There is described a solid reservoir reference electrode having a first layer of a metal; a second layer of a salt of the metal and a non-metallic species atop the first layer; a third layer of an electrolyte salt atop the second layer, the electrolyte salt including the non-metallic species; and a fourth layer of an inert polymer atop the third layer; wherein the electrolyte salt is soluble in an electrolytic solution receivable atop the fourth layer; the fourth layer, when contacted with the electrolyte solution, has first channels allowing the electrolytic solution to flow through the fourth layer; the third layer has second channels formed therein upon dissolution of the electrolyte salt by the electrolytic solution flowing through the first channels of the fourth layer; and the electrolytic solution is in fluid communication with the salt of the metal of the second layer through the first and second channels.

### TECHNICAL FIELD

This disclosure generally relates to electrode potential stability and more particularly relates to reference electrodes.

### BACKGROUND OF THE ART

In electrochemistry, reference electrodes are used to provide access to a stable and well-defined electrochemical potential of a liquid electrolytic solution. Reference electrodes are useful in a variety of electrochemical applications, including electrodeposition, electrolysis, and potentiometric sensing. A conventional Ag/AgCl reference electrode is generally made of a bulky glass structure containing a chlorinated Ag wire immersed in a highly concentrated (e.g., 3 M to 4.5 M) aqueous potassium chloride (KCl) solution, terminated with a porous frit. The immersion of the reference electrode into the solution allows for the KCl solution to establish an equipotential with the surrounding aqueous electrolyte by ionic conduction. Aqueous KCl leaks from the finite reservoir within the electrode through the frit, as a necessity to establishing an equipotential with the environment. As can be inferred from the redox half-reaction, the electrode potential is dependent upon the chloride ion (Cl⁻¹) concentration. A stable reference potential thus requires a stable Cl⁻ concentration within the reservoir. The performance of a reference electrode is generally associated with the stability of its electric potential in the electrolyte solution and the environment. Improvements in the stability and performance of reference electrodes are therefore desired.

### SUMMARY

The present disclosure provides a solid reservoir reference electrode that at least achieves the performance of traditional immersed wire reference electrodes but in a compact, layered structure. The solid reservoir reference electrode achieves the simultaneous combination of electric potential stability over time, independent of the chemical environment, with a simple layered structure that is more compact than traditional immersed wire reference electrodes. The solid reservoir reference electrode has improved stability compared to other layered reference electrode structures. In some embodiments, the solid reservoir reference electrode has an improved shelf life compared to traditional immersed wire reference electrodes. Further, the solid reservoir reference electrode is a layered electrode that can be manufactured more easily and cost effectively compared to the traditional immersed wire reference electrodes.

In accordance with a first aspect of the present disclosure, there is provided a solid reservoir reference electrode comprising: a first layer of a given metal; a second layer of a salt of the given metal and a non-metallic species atop the first layer; a third layer of an electrolyte salt atop the second layer, the electrolyte salt including the non-metallic species; and a fourth layer of an inert polymer atop the third layer; wherein the electrolyte salt is soluble in an electrolytic solution receivable atop the fourth layer; the fourth layer, when in contact with the electrolyte solution, has first channels allowing the electrolytic solution to flow through the fourth layer; the third layer has second channels formed therein upon dissolution of the electrolyte salt by the electrolytic solution flowing through the first channels of the fourth layer; and the electrolytic solution is in fluid communication with the salt of the given metal of the second layer through the first channels and the second channels.

Further in accordance with the first aspect of the present disclosure, the inert polymer can for example be porous and can for example have the first channels incorporated therein.

Still further in accordance with the first aspect of the present disclosure, the inert polymer can for example have a sacrificial material incorporated therein, the sacrificial material can for example be soluble in the electrolytic solution and when the inert polymer comes into contact with the electrolytic solution, the first channels can for example be formed therein upon dissolution of the sacrificial material.

Still further in accordance with the first aspect of the present disclosure, the third layer can for example include a crystalline, a polycrystalline, and/or a powdered form of the electrolyte salt.

Still further in accordance with the first aspect of the present disclosure, the electrolyte salt can for example have a grain size of less than 20 μm.

Still further in accordance with the first aspect of the present disclosure, the given metal can for example be selected from Ag, Cu, Zn, Au, Pt, Al, Cr, Ni, Sn, Fe, Co, oxides thereof and doped alloys thereof.

Still further in accordance with the first aspect of the present disclosure, the non-metallic species can for example be selected from Cl⁻, Br⁻, F⁻, I⁻, SO₄²⁻, S²⁻, OH⁻, CO₃²⁻, PO₄³⁻, HCO₃⁻, HPO₄²⁻, H₂PO₄⁻, PF₆⁻, BF₄⁻, NO₃⁻, a formate anion, an acetate anion, an oxalate anion, a citrate anion, ethylenediaminetetraacetic acid (EDTA), a sulfonate anion, dicyanamide, bistriflimide, a cyclopentadienyl anion and derivatives thereof, and a pyridine anion and derivatives thereof.

Still further in accordance with the first aspect of the present disclosure, the electrolyte salt can for example be selected from NaCl, Na₂SO₄, (NH₄)Cl, LiCl, RbCl, CaCl₂, KCl, MgCl₂, tetraethylammonium chloride (TEACl), tetrabutylammonium chloride (TBACl), K₂SO₄, Na₂S, or K₂S.

Still further in accordance with the first aspect of the present disclosure, the electrolyte salt can for example be a halogen containing salt and the salt of the given metal is a salt of the given metal and the halogen.

Still further in accordance with the first aspect of the present disclosure, the electrolyte salt can for example be KCl, NaCl, CaCl₂, or MgCl₂.

Still further in accordance with the first aspect of the present disclosure, the salt of the given metal can for example be Ag₂S, CuS, CuSO₄, Ag₂SO₄, CuCl₂, AgCl, NiS, NiSO₄, NiCl₂, AuS, Au₂SO₄, AuCl, PtS, PtSO₄ or PtCl₂.

Still further in accordance with the first aspect of the present disclosure, the electrolyte salt can for example be K₂S, Na₂S, Na₂SO₄ or K₂SO₄.

Still further in accordance with the first aspect of the present disclosure, a mass ratio of the inert polymer to the sacrificial material incorporated therein can for example range from 0.1 to 1.

Still further in accordance with the first aspect of the present disclosure, the inert polymer can for example be selected from polydimethylsiloxane (PDMS), polyethylene terephthalate (PET), poly(vinyl alcohol) (PVA), poly(vinyl butyral) (PVB), poly(vinyl chloride) (PVC), and thermoplastic polyurethane (TPU).

Still further in accordance with the first aspect of the present disclosure, the electrolytic solution can for example be an aqueous solution and the sacrificial material incorporated in the inert polymer is hygroscopic.

Still further in accordance with the first aspect of the present disclosure, the sacrificial material can for example be a saccharide, a salt or the electrolyte salt.

Still further in accordance with the first aspect of the present disclosure, the solid reservoir reference electrode can for example further comprise a substrate layer supporting the first layer.

Still further in accordance with the first aspect of the present disclosure, the substrate layer can for example include silicon, glass, alumina, fiberglass, polyethylene terephthalate (PET), PDMS, thermoplastic polyurethane (TPU), polyethylene terephthalate glycol (PETG), acrylonitrile butadiene styrene (ABS), silica, alumina, nylon, and polylactic acid (PLA).

In accordance with a second aspect of the present disclosure, there is provided a potentiometric sensor comprising the reference electrode as disclosed herein, and at least one of a working electrode and an ion sensitive field effect transistor.

Further in accordance with the second aspect of the present disclosure, the potentiometric sensor can for example be one of a biosensor, a wrist strap, and a test strip.

Still further in accordance with the second aspect of the present disclosure, the biosensor can for example be at least one of an electroencephalogram sensor and an electrocardiogram sensor.

In accordance with a third aspect of the present disclosure, there is provided an array of potentiometric sensors comprising the reference electrode as disclosed herein and at least one of an array of working electrodes and ion sensitive field effect transistors.

In accordance with a fourth aspect of the present disclosure, there is provided a solid reservoir reference electrode comprising: a first layer of a given metal; a second layer of a salt of the given metal and a non-metallic species atop the first layer; a third layer of an electrolyte salt atop the second layer, the electrolyte salt including the non-metallic species; and a fourth layer of an inert polymer atop the third layer.

Further in accordance with the second aspect of the present disclosure, the inert polymer can for example be porous.

Still further in accordance with the fourth aspect of the present disclosure, the inert polymer can for example have a sacrificial material incorporated therein.

All technical implementation details and advantages described with respect to a particular aspect of the present disclosure are self-evidently mutatis mutandis applicable for all other aspects of the present disclosure.

Many further features and combinations thereof concerning the present improvements will appear to those skilled in the art following a reading of the instant disclosure.

### DESCRIPTION OF THE DRAWINGS

In the figures,

FIG. 1 is a schematic side elevation cross section view of an example of a solid reservoir reference electrode, in accordance with one or more embodiments;

FIG. 2 is an optical microscopy image of an exemplary first layer of a solid reservoir reference electrode containing polydimethylsiloxane (PDMS) with KCl (PDMS/KCl) incorporated therein at a mass ratio of 1:1, in accordance with one or more embodiments;

FIG. 3 is a graph showing the open circuit potential over time measured in deionized water with a reference electrode according to an embodiment of the present disclosure ( [symbol] ) and a reference electrode according to the prior art ( [symbol] );

FIG. 4 is a graph showing the open circuit potential over time measured in changing concentration of aqueous NaCl with a reference electrode according to an embodiment of the present disclosure ( [symbol] ) and a AgCl solid layer ( [symbol] );

FIG. 5 is a graph showing the open circuit potential over time measured in changing concentration of aqueous KCl with a reference electrode according to an embodiment of the present disclosure ( [symbol] ) and a AgCl solid layer ( [symbol] );

FIG. 6 is a graph showing the open circuit potential over time measured in changing pH buffers (4, 7, and 10) with a reference electrode according to an embodiment of the present disclosure ( [symbol] ) and a AgCl solid layer ( [symbol] );

FIG. 7 is a graph showing the open circuit potential over time measured in saturated KCl aqueous concentration with a reference electrode according to an embodiment of the present disclosure having a PDMS/KCl layer having a thickness of 0.2 mm ( [symbol] ) and 1 mm ( [symbol] );

FIG. 8 is a graph showing the pH sensitive field effect transistor current versus reference potential in changing pH buffers (4, 7, 10) with a reference electrode according to an embodiment of the present disclosure;

FIG. 9 is a graph showing the pH sensitive field effect transistor neutrality potential versus pH with a reference electrode according to an embodiment of the present disclosure;

FIG. 10 is a graph showing the Na⁺ sensitive field effect transistor current versus reference potential in NaCl solutions of changing concentration (10⁻¹ M, 10⁻² M, 10⁻³ M) with a reference electrode according to an embodiment of the present disclosure;

FIG. 11 is a graph showing the Na⁺ sensitive field effect transistor neutrality potential versus logarithm of Na⁺ concentration with a reference electrode according to an embodiment of the present disclosure;

FIG. 12 is a graph showing the current versus potential of an electrochemical cell after different durations of operation (1, 2, 3 and 4 hours) with non-aqueous acetonitrile solvent, ferrocene/ferrocenium cations, Pt working electrode, graphite counter-electrode, and a reference electrode according to an embodiment of the present disclosure;

FIG. 13 is a graph showing the mean of the ferrocene/ferrocenium redox potentials versus time in raw ( [symbol] ) and moving average ( [symbol] ) for an electrochemical cell with non-aqueous acetonitrile solvent, ferrocene/ferrocenium cations, Pt working electrode, graphite counter-electrode, and a reference electrode according to an embodiment of the present disclosure;

FIG. 14 is a graph of the current in function of the potential for a voltametric sweep of the ferrocenium ion/ferrocene (fc⁺/fc) redox couple with SRRE as the reference electrode;

FIG. 15 is a graph of potential over time showing the inferred shift in the potential of the SRRE reference electrode while in acetonitrile;

FIG. 16 is an electrocardiogram of a forearm performed with a disposable gel electrode or a solid reservoir reference electrode (SRRE);

FIG. 17 is an electroencephalogram taken from the forehand to the back of the head performed with a disposable gel electrode or a SRRE; and

FIG. 18 is a graph showing the background noise measured for the electroencephalogram of FIG. 17.

### DETAILED DESCRIPTION

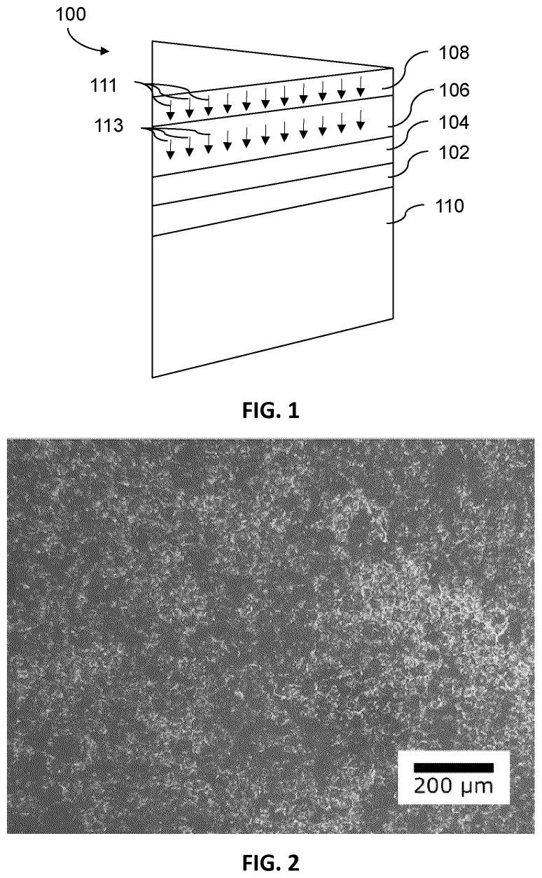

FIG. 1 shows an example of a solid reservoir reference electrode 100. As depicted, the solid reservoir reference electrode 100 is a multilayer electrode. More specifically, the solid reservoir reference electrode 100 includes a first layer 102 of a given metal, a second layer 104 of a salt of the given metal atop the first layer 102, a third layer 106 of an electrolyte salt atop the second layer 104, and a fourth layer 108 of an inert polymer atop the third layer 106. The solid reservoir reference electrode 100 is optionally supported by a substrate layer 110. In other words, the first layer 102 can be atop the substrate layer 110 in some embodiments. However, in some other embodiments, the substrate layer 110 can be omitted.

The inert polymer is porous or becomes porous when coming into contact with the electrolytic solution. In other words, the inert polymer has first channels formed therein before contacting the electrolytic solution or can form the first channels upon dissolution of a sacrificial material incorporated therein. In FIG. 1, the first channels are schematically illustrated via arrows 111 extending within the fourth layer 108 of the inert polymer. There are many methods for creating a porosity in an inert polymer to obtain a porous polymer in the fourth layer 108. In some embodiments, the inert porous polymer can be obtained through mixing the inert polymer precursor with a solvent that would dissolve the precursor. For example, if polydimethylsiloxane (PDMS) is the inert polymer, the solvent can be tetrahydrofuran (THF) or toluene. The evaporation of the THF or toluene from the precursor/solvent mixture allows for the formation of pores in the final inert polymer. The ratio of precursor to solvent can be modified to increase or decrease the porosity. The method of depositing the fourth layer 108 can be chosen accordingly and includes but is not limited to screen printing, inkjet printing and drop cast. In embodiments where the inert polymer is not porous but becomes porous upon contacting the electrolyte solution, the inert polymer contains a sacrificial material incorporated therein. Both the electrolyte salt and the sacrificial material are soluble in an electrolytic solution receivable atop the fourth layer 108. For instance, the electrolytic solution can be poured, immersed or flowed atop the fourth layer 108 during use of the solid reservoir reference electrode 100. Equivalently, the solid reservoir reference electrode may be immersed or brought into contact with the electrolytic solution, or with a material bearing an electrolytic solution. Accordingly, first channels are formed in the fourth layer 108 upon dissolution of the sacrificial material by the electrolytic solution.

Second channels are formed in the third layer 106 therein upon dissolution of the electrolyte salt by the electrolytic solution flowing through the first channels of the fourth layer 108. As shown, the second channels are schematically illustrated via arrows 113 extending within the third layer 106. Therefore, after the formation of the second channels, the electrolytic solution becomes in fluid communication with the salt of the given metal of the second layer 104 through the first channels and the second channels. By being in fluid communication with the second 104 layer, the electrolyte salt in the third layer 106 acts as a reservoir of electrolyte salt to continuously replenish the supply of electrolyte salt at the interface of the reference electrode and maintain a sufficient and constant concentration of electrolyte to improve the performance of the reference electrode including stabilizing its potential.

By not being in fluid communication with the external environment until the first layer is contacted with a solution that dissolves the salt in the fourth layer 108, the second layer 104 and in some cases the third layer 106 (and their contents) are preserved before use and are protected from the oxidizing conditions of ambient air as well as other external environment conditions. In embodiments where the fourth layer 108 is not porous and has the capacity of forming pores when contacted with the electrolytic solution, the preservation of the content of the second and third layers 104 and 106 can improve the shelf life of the solid reservoir reference electrode by having the fourth layer 108 acting as both a protective layer during storage and a separation layer to establish an equipotential with the surrounding electrolytes by ionic conduction when the solid reservoir reference electrode is in use (e.g., akin to the porous frit in a traditional immersed wire reference electrode). This can allow for the establishment and maintenance of the equipotential with the external environment, while locally saturating the concentration of electrolyte salt near or at the interface of the second layer 104 where the redox reaction occurs.

The solid reservoir reference electrode of the present disclosure can be used in any potentiometric sensor where a reference electrode is required. Accordingly, the given metal can be selected based on the specific application of the potentiometric sensor. The given metal is a generally a conductive metal that is different from the material used in a working electrode of the potentiometric sensor. In some embodiments, the given metal is a conductive cationic metal. In some embodiments, the given metal is selected from Ag, Cu, Zn, Au, Pt, Al, Cr, Ni, Sn, Fe, Co, oxides thereof and doped alloys thereof, to name a few examples. Silver (Ag) is one of the most commonly used metals for solid reservoir reference electrodes due to a combination of good performance and cost effectiveness. In some embodiments, silver is the preferred given metal.

The potentiometric sensor can determine the presence, the absence, the change or the specific concentration of an analyte by comparing the electrical reading (e.g. electric potential) at the working electrode with the reading at the reference electrode. Therefore, the role of the reference electrode is to maintain a constant reading that is independent from the presence, absence or concentration of the analyte. The analyte changes the reading at the working electrode which allows its detection. Non-limitative examples of the analyte include a molecule (e.g., biomolecule), a pH, an ion or a contaminant (for example in the context of waste water treatment). The selection of the given metal and the composition of the working electrode can vary based on the analyte.

The second layer 104 contains a salt of the given metal. The salt of the given metal is the redox couple of the given metal and an appropriate salt formed by the given metal and a non-metallic species. The non-metallic species may be a halogen. The non-metallic species can be selected from Cl⁻, Br⁻, F⁻, I⁻, SO₄²⁻, S²⁻, OH⁻, CO₃²⁻ , HCO₃⁻, PO₄³⁻, HPO₄²⁻, H₂PO₄⁻, NO₃⁻, PF₆⁻, and BF₄⁻. The non-metallic species can be selected from organic anions and organic ligands. Examples of organic anions and organic ligands include carboxylate species-such as formates, acetates, oxalates, citrates, and ethylenediaminetetraacetic acid (EDTA)—sulfonates, dicyanamide, bistriflimide, derivatives of the cyclopentadienyl anion, and derivatives of pyridine, such as 2,2′-bipyridine. Examples of a redox couple include but are not limited to Ag⁰/Ag⁺ with the salt AgCl, AgNO₃, Ag₂SO₄, or Ag₂S, Cu⁰/Cu²⁺ with the salt CuSO₄, CuCl₂, or CuS, Ni⁰/Ni²⁺ with the salt NiS, NiSO₄ or NiCl₂, Au⁰/Au⁺ with the salt AuS, Au₂SO₄ or AuCl, Pt⁰/Pt²⁺ with the salt PtS, PtSO₄ or PtCl₂. The potential at the metal-solution interface at the second layer 104 is generally set by the reversible redox half-reaction between the given metal and the metal cation, and by the concentration of the non-metallic anionic species of the salt. An exemplary reaction is shown below for a monoelectronic redox half reaction where “A” is the given metal and “B” is the non-metallic species forming the salt of the second layer 104 with the given metal:

$$A {B}_{s} + {e}^{-} ↔ {A}_{( s )} + {B}_{( a q )}^{-} .$$

During operation of the potentiometric sensor, the potential of the working electrode or ion sensitive field effect transistor can be more positive, equal, or more negative than that of the reference electrode in use. When the working electrode or ion sensitive field effect transistor is more positive, electrons flow from the reference electrode to the working electrode or ion sensitive field effect transistor. These electrons are released by the given metal in the first layer 102 in contact with the second layer 104, which is partially oxidized to its salt and becomes part of the second layer 104, after combining with the free, non-metallic species of the salt (corresponding redox semi-reaction: A+B⁻→AB+e⁻). When the working electrode or ion sensitive field effect transistor is more negative, electrons flow from the working electrode or ion sensitive field effect transistor to the reference electrode. These electrons are captured by the cations/metallic species of the salt of the given metal in the second layer 104 in contact with the first layer 102, which are partially reduced to the native state (0) of the metal and become part of the first layer 102 (corresponding redox semi-reaction: AB+e⁻→A+B⁻). As can be inferred from the redox half-reaction, the electrode potential of the solid reference electrode is dependent upon the non-metallic ion species (e.g., B⁻ or B²⁻ ) concentration. A stable reference potential thus requires a stable B ion concentration throughout the use of the potentiometric sensor. This is achieved in the present solid reference electrode with the third layer 106 which contains an electrolyte salt to replenish the non-metallic species.

The third layer 106 can be considered to contain a solid reservoir of the electrolyte salt. The electrolyte salt is a salt of the non-metallic species and preferably a cationic species. Examples of the cationic species include but are not limited to Na⁺, K⁺, Rb⁺, NH₄⁺, Li⁺, Ca²⁺, Mg²⁺, or Sr²⁺, to name a few examples. The cationic species can be organic ions including pyrrolidinium, pyridinium, imidazolium, phosphonium, and ammonium derivatives. Examples of electrolyte salt include but are not limited to NaCl, Na₂SO₄, (NH₄)Cl, LiCl, RbCl, CaCl₂, KCl, MgCl₂, tetraethylammonium chloride (TEACl), tetrabutylammonium chloride (TBACl), K₂SO₄, Na₂S, or K₂S. The third layer 106 provides the supply of electrolyte salt which allows a constant saturated concentration of electrolyte at the interface where the redox reaction occurs. The third layer 106 therefore allows the reference electrode to maintain an electric potential that is constant over time, until the solid reservoir is entirely consumed. Compared to prior art reference electrodes such as the conventional Ag/AgCl reference electrode described in US2020011664 without the layer 106, the prior art's electrical potential continuously drifts over time due to the depletion of chloride ions. Another advantage of having a reservoir of electrolyte salt in close proximity or adjacent to the second layer 104 is that the solid reservoir electrode can maintain a potential that is independent of the chemical environment by creating a local concentrated or saturated environment at the interface.

In some embodiments, the electrolyte salt is in crystalline, polycrystalline, and/or a powdered form. The electrolyte salt can have a grain size of less than 100 μm, less than 50 μm, less than 30 μm, less than 20 μm or less than 10 μm, depending on the embodiment. A smaller grain size allows for an easier dissolution of the electrolyte salt in the electrolytic solution and therefore a better and more rapid formation of the second channels in the third layer. The use of smaller grain size allows for a much thinner 108 layer, to reduce the electrode's resistance and allow for a wider range of membrane deposition techniques such as spray coating and inkjet printing. As explained above, the electrolyte salt is soluble in the electrolytic solution and therefore when contacted with the electrolytic solution second channels are formed across and through the third layer 106 that allow the passage of ions from the electrolytic solution to the second layer 104.

To separate the third layer 106 from the external environment and the electrolytic solution such that the third layer can act as a reservoir of electrolyte salt and create a local environment, a fourth layer 108 of inert polymer is atop the third layer 106. The inert polymer is inert with respect to the electrochemical reaction occurring at the reference electrode and preferably does not interfere with the redox reaction occurring at the reference electrode. The inert polymer can be selected from polydimethylsiloxane (PDMS), polyethylene terephthalate (PET), poly(vinyl alcohol) (PVA), poly(vinyl butyral) (PVB), poly(vinyl chloride) (PVC), and thermoplastic polyurethane (TPU).

The inert polymer can be provided as a porous polymer or a sacrificial material can be incorporated in the fourth layer to create first channels in the inert polymer when the inert polymer is exposed to the electrolytic solution. Accordingly, the sacrificial material is soluble in the electrolytic solution. In some embodiments, the electrolytic solution is aqueous and the sacrificial material is preferably hygroscopic. The present disclosure also contemplates non-aqueous electrolytic solvents such as acetonitrile. In some examples, the sacrificial material can be a saccharide (sugar) or other soluble molecule. Additionally or alternatively, the sacrificial material can be a salt such as the electrolyte salt. In some embodiments, it is preferred that the sacrificial material is the electrolyte salt in order to reduce the introduction of contaminants in the electrode. The selection of the sacrificial material can determine the size of the first channels formed in the inert polymer. For example, KCl or NaCl generally form pores smaller that those obtained with a sugar. Although the salt used as the sacrificial material can be the electrolyte salt, other suitable salts are also contemplated herein such as Na₂CO₃.

The number of pores and porosity level of the inert polymer can be controlled by the amount of sacrificial material added in the inert polymer or by the method used for making the porous inert polymer. In some embodiments, a weight ratio of from 12:1 to 3:1 of inert polymer to sacrificial material can be used, preferably from 10:1 to 5:1. The stiffness of the inert polymer depends on the porosity and the stiffness can therefore be controlled by controlling the weight ratio of inert polymer to sacrificial material. An increased stiffness can improve the response time of the reference electrode and accelerate the assay time of an electrochemical assay using the solid reservoir electrode of the present disclosure.

The thickness of the first, second, third and fourth layers 102, 104, 106 and 108 can be adapted depending on the use of the reference electrode and the corresponding scale. For example, a microfluidic device will have micron range thicknesses whereas a larger electrical device can have macro range thickness in the order of millimetres. Without wishing to be bound by theory, the first layer 102 and the third layers 106 may have a thickness that is as large as the specific application of the solid reference electrode allows, the thickness of the second layer 104 is preferably just thick enough to provide an electron-ion charge transfer interface, and the fourth layer 108 should be thick enough to provide the desired covering of the third layer but not too thick such that ion conduction is too slow or prevented. In some embodiments, the thickness of the fourth layer 108 has an ion conduction of around 10 kohms or at least 10 kohms.

The solid reference electrode 100 is optionally supported by a substrate layer 110. In some embodiments, the substrate layer 110 comprises silicon, glass, alumina, fiberglass, polyethylene terephthalate (PET), PDMS, thermoplastic polyurethane (TPU), polyethylene terephthalate glycol (PETG), acrylonitrile butadiene styrene (ABS), silica, alumina, nylon, and polylactic acid (PLA), to name a few examples. The substrate layer 110 can be inert with respect to the electrochemical reaction and serves to provide physical support. The substrate layer is preferably non-conductive. In some embodiments, the substrate layer 110 is part of an electrochemical detection device (for example the base of a microfluidic device or a test strip).

The solid reservoir reference electrode 100 can optionally be fabricated on the substrate layer 110 and the substrate layer 110 may be a sacrificial substrate layer such that the solid reservoir reference electrode is incorporated in the potentiometric sensor without the substrate layer 110. The solid reservoir reference electrode 100 can accordingly be fabricated by providing the given metal or by depositing the given metal on the substrate layer 110. The first layer 102 can be deposited as a paint followed by curing at room temperature or at elevated temperature. For example, Ag flakes suspended in acrylic based lacquer can be applied and cured at room temperature. In another example, Ag powder in butyl acetate or in mixtures of ketones can be applied and cured at room temperature or elevated temperature up to 100° C. The first layer 102 can also be deposited by powder coating, electrodeposition, electroless deposition, doctor blading, screen printing, roll-to-roll printing, gravure printing, inkjet printing, aerosol printing, spray coating, thermal evaporation, electron-beam evaporation, laser ablation, plasma deposition, 3D printing or any other suitable means. The second layer 104 is then deposited on top of the first layer 102. The second layer 104 can be physically deposited as a paint followed by curing at room temperature or at high temperature (e.g. 80-120° C. but depends on the given metal selected). The second layer 104 can also be deposited by powder coating, electrodeposition, electroless deposition, doctor blading, screen printing, roll-to-roll printing, gravure printing, inkjet printing, aerosol printing, spray coating, thermal evaporation, electron-beam evaporation, laser ablation, plasma deposition, 3D printing or any other suitable means. The electrolyte salt is placed on top of the second layer 104 to form the third layer 106 which may be in powder form. Optionally, the powder of electrolyte salt is ground or milled to a desired grain size before depositing the powder on the second layer 104. The inert polymer can be deposited onto the third layer 106 and cured under conditions that would render the inert polymer porous or the inert polymer can contain the sacrificial material to form the fourth layer 108. The sacrificial material in layer 108 can be the electrolytic salt of layer 106, or another sacrificial material. For example, for manufacturing methods such as screen printing that require high viscosity of the deposited material a sacrificial material such as sugar may be used. For manufacturing methods that require lower viscosity of the deposited material, the sacrificial material can be a solvent such as tetrahydrofuran, toluene or acetone which does not dissolve the electrolytic salt in the layer 106 below. The curing temperature can be room temperature or a higher temperature, such as 70-90° C. but depends on the selected inert polymer. For example, PDMS can be cured at room temperature.

In another aspect, there is provided an potentiometric sensor comprising the solid reservoir reference electrode 100 and a working electrode (not shown) or ion sensitive field effect transistor. The sensor can be a pH sensor, an ion-sensor, a biosensor, a wrist strap, a test strip, an electroencephalogram or an electrocardiogram, for instance. The solid reservoir reference electrode has an applied potential difference relative to the potential of a working electrode or ion sensitive field effect transistor in order to determine the presence, absence or concentration of various analytes. Indeed, advantages of the present solid reservoir reference electrode is its independence on the electrochemical composition of the electrolytic solution and a compact layered structure that enables miniaturization, monolithic integration with other elements such as sensors, incorporation in flexible elements such as wrist straps, and compatibility with a wide variety of manufacturing methods.

In another aspect, there is provided an array of potentiometric sensors comprising the solid reservoir reference electrode 100 and an array of working electrode (not shown) or ion sensitive field effect transistors in a combination suitable to the desired detection and/or measurement of multiple analytes.

Conventional Ag/AgCl immersed wire reference electrodes cannot be used in non-aqueous solvents/as the dissolved KCl has a tendency to precipitate in non-aqueous solvents, clogging the system at the electrode's frit. The precipitation occurs due to the KCl's transition from a high-solubility to a low-solubility environment. As no such transitions occur in the solid reservoir reference electrode of the present disclosure, it can be used directly in non-aqueous solvents, such as acetonitrile.

### EXAMPLE

The solid reservoir reference electrode (SRRE) was fabricated by painting Ag and AgCl ink onto a glass substrate and curing at 100° C. on a hotplate for 1 hour. KCl powder was ground in a mortar with a pestle and was packed on top of the AgCl layer. To encapsulate the KCl powder, a 1:1 by weight mixture of PDMS and powdered KCl was painted on top with a steel blade. The PDMS directly incorporated the fine powder of solid KCl having an average grain diameter of less than 20 μm to form a PDMS/KCl layer. An optical microscope image of the PDMS/KCl layer is shown in FIG. 2. A porous surface structure can be observed after exposure to an aqueous solution.

Due to KCl's high solubility and hygroscopic nature, when exposed to an aqueous solution such as water, pores readily formed in the PDMS/KCl layer and aqueous ionic channels (i.e. the second channels). formed in the solid powder KCl layer. This allowed for the rapid establishment of an equipotential with the external environment, while locally saturating the concentration of chloride near the AgCl layer. The solid powder phase of the KCl reservoir enabled a Cl ion density storage of 13.4 mmol/cm³ approximately 3.0 times denser than a saturated KCl aqueous phase reservoir.

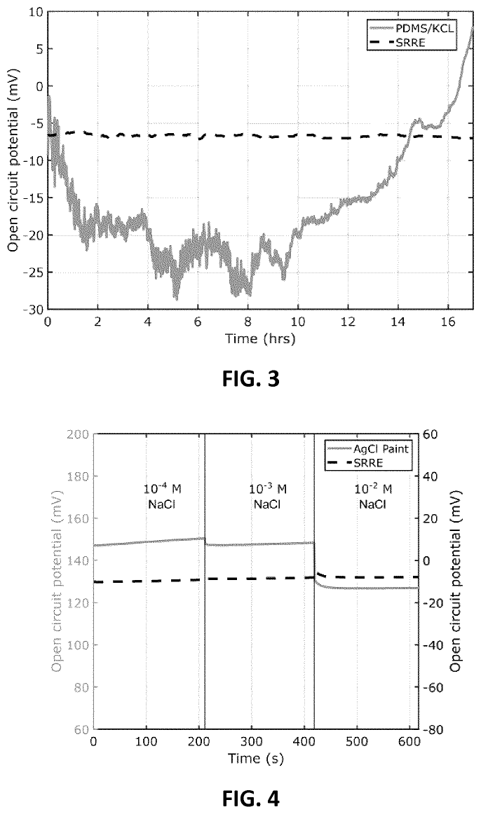

An ideal reference electrode should exhibit an electrochemical potential that: 1) does not vary with time, and 2) is independent of the chemical environment (e.g. changes in ion concentrations within the electrolyte). The electrochemical potential of the reference electrode was quantified through measuring the open circuit potential (OCP) versus a commercially available Ag/AgCl/KCl (3 M) bulk reference electrode, BASi MF-2056. In FIG. 3, the OCP of the SRRE in the harsh environment of deionized (DI) water is plotted as a dashed line. For comparison, the OCP of a reference electrode according to US20200116664 in DI water was plotted as a solid line. This prior art PDMS based reference electrode differs from the SRRE in that it is composed solely of a PDMS/KCl layer with no second layer as described with respect to FIG. 1. In other words, the KCl solid salt layer is included in the present SRRE but absent in the comparative prior art reference electrode. Over 17 hours, the SRRE's OCP varied by less than 2 mV while the OCP measured for the comparative prior art reference electrode varied more than 30 mV, and became increasingly unstable after 17 hours. This result demonstrates the SRRE's improved temporal stability.

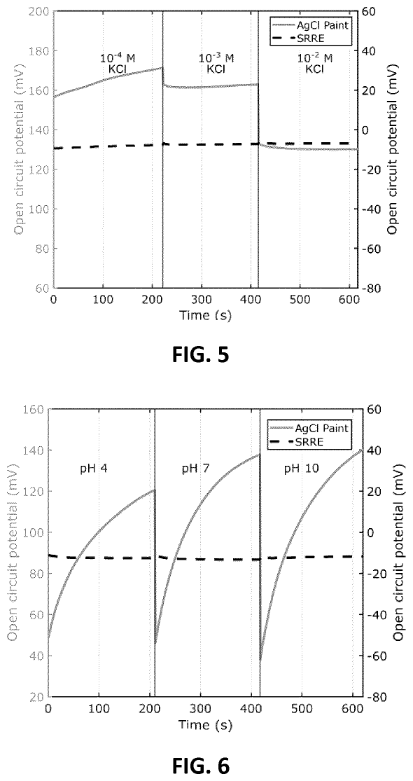

In FIGS. 4 and 5, the OCP of the SRRE, was plotted as a dashed line and was compared to the OCP of a Ag/AgCl paint layer, plotted as a solid line, when immersed in aqueous solutions with different concentrations of NaCl and KCl. While the SRRE's OCP varied less than 3 mV, the silver paint layer's OCP varied more than 20 mV between 10⁻⁴ M to 10⁻² M of NaCl and KCl. In FIG. 6, the OCP of the SRRE, was plotted as a dashed line and was compared to the OCP of a Ag/AgCl paint layer, plotted as a solid grey line, when immersed in buffer solutions with pH values of 4, 7, and 10. The OCP measured for the Ag/AgCl paint drifted significantly (up to 100 mV) due to the low concentration of chloride ions in the buffer solutions, while the OCP measured for the SRRE remained stable within a range of 2 mV, demonstrating the SRRE's insensitivity to the chemical environment.

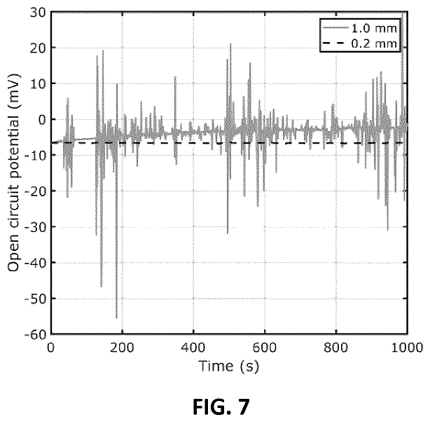

In FIG. 7, the role of the thickness of the PDMS/KCl layer for the reference electrode was evaluated. The OCP of two SRREs in a saturated KCl solution was measured, one with a 1-mm thickness (solid line) and one with a 0.2-mm thickness (dashed line) layer of PDMS/KCl. Significant OCP fluctuation can be observed for the SRRE with a 1-mm thick layer of PDMS/KCl, which is attributed to the high ionic impedance of the thick PDMS/KCl layer. A more stable OCP is established with the use of a thinner, and thus less impeding, PDMS/KCl layer.

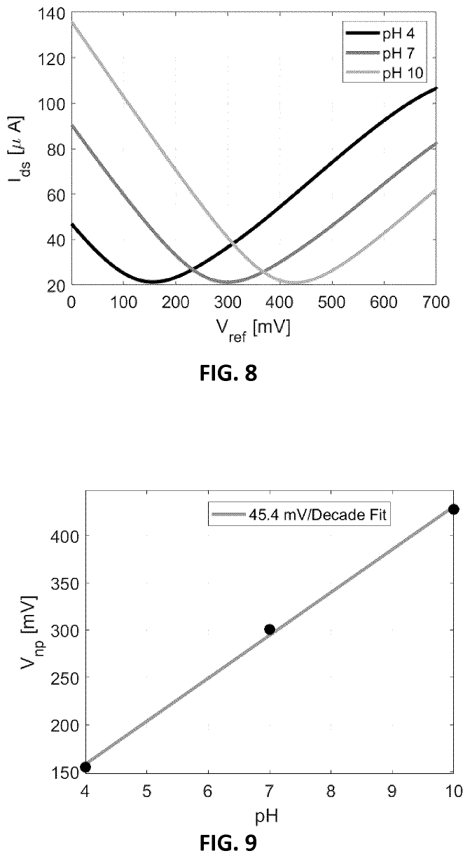

FIGS. 8 and 9 demonstrate the performance of the SRRE as the reference electrode for a pH sensor with a pH sensitive graphene field effect transistor. The SRRE and sensor were immersed in electrolytic solutions of known pH. The sensor current was measured versus the potential applied between the sensor and the SRRE. The sensor current versus applied potential depends on pH. In FIG. 9, the potentiometric sensitivity of the pH sensor was evaluated by plotting the applied potential at which minimum sensor current was observed versus pH (identified from current versus applied potential in FIG. 8).

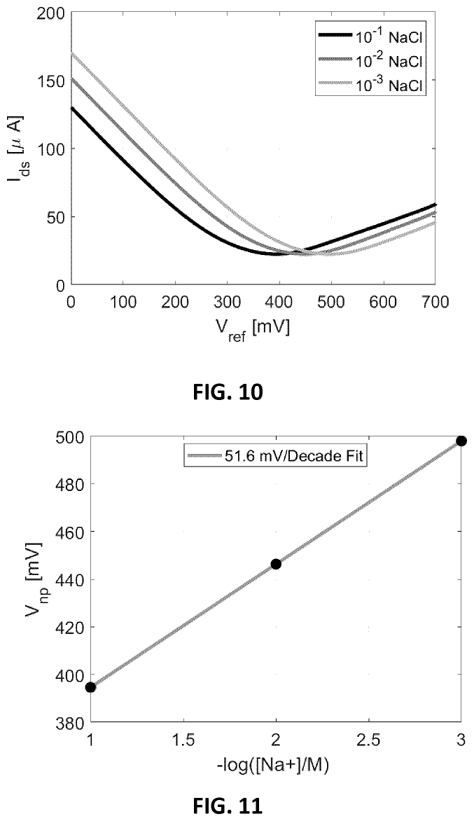

FIGS. 10 and 11 demonstrate the performance of the SRRE as the reference electrode for a Na⁺ ion sensor with a Na⁺ ion sensitive graphene field effect transistor. The SRRE and sensor were immersed in electrolytic solutions of known NaCl concentration. The concentration of Nations was determined by the NaCl concentration. The sensor current was measured versus the potential applied between the sensor and the SRRE. The sensor current versus applied potential varied with NaCl concentration. In FIG. 11, the potentiometric sensitivity of the pH sensor was evaluated by plotting the applied potential at which minimum sensor current is observed versus the logarithm of Na⁺ concentration (identified from current versus applied potential in FIG. 8).

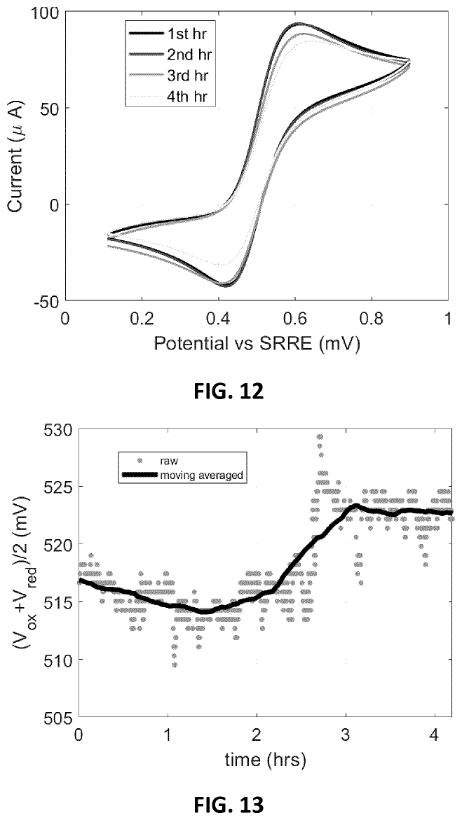

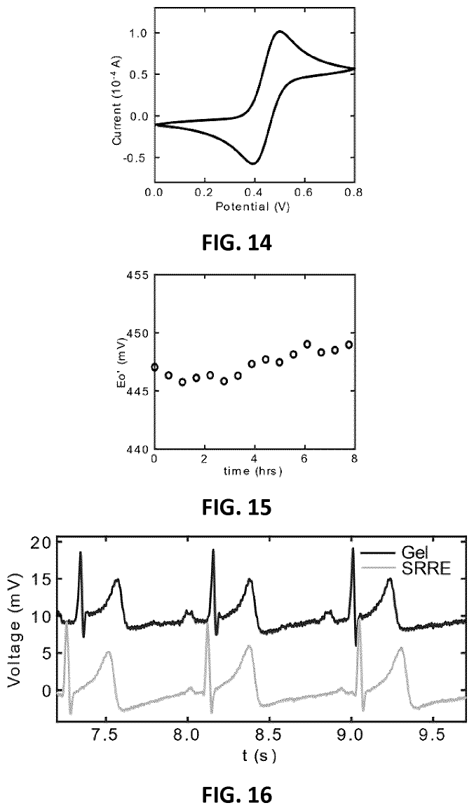

FIGS. 12-15 demonstrate the stability of the SRRE potential in a non-aqueous environment. The SRRE potential was immersed in an acetonitrile-based electrolyte (0.1 M tetrabutylammonium hexafluorophosphate) containing ferrocene (1 mM). The SRRE potential was evaluated by recording a voltammetric cycle where the potential of a Pt sheet working electrode was swept relative to the SRRE potential (FIG. 12). The symmetric voltammograms show the fully reversible oxidation and subsequent reduction of the ferrocenium ion/ferrocene (fc⁺/fc) redox couple. The mean value of the potential of the oxidative and the reductive peaks of current relate to the fc⁺/fc couple, i.e. the fc⁺/fc redox potential measured at the working electrode, allows one to estimate the SRRE potential. The absolute redox potential of the fc⁺/fc couple in acetonitrile is known (E0=0.400 V vs SHE). The stability of the SRRE potential over 4 hours was assessed by recording multiple subsequent voltammetric cycles and plotting the mean value of the measured potentials of the oxidative and the reductive peaks of the fc⁺/fc couple versus time (FIG. 13). The redox reaction between fc⁺ and fc was highly reversible, and thus every shift in the measured fc⁺/fc redox potential over time can be ascribed to a corresponding change of the SRRE potential. As shown in FIG. 13, the SRRE potential (moving average) was remarkably stable over time in a non-aqueous environment, shifting less than 10 mV over a 4-hour timespan. FIG. 14 shows the 1^{st} voltametric sweep of fc+/fc in acetonitrile with the SRRE as the reference electrode, in conjunction with a graphite working electrode and a graphite counter electrode. FIG. 15 shows the nferred shift in the potential of the SRRE reference electrode while in acetonitrile, a non-aqueous solvent. Significant stability of the SRRE was observed in acetonitrile with a drift of less than 2 mV over 8 hours.

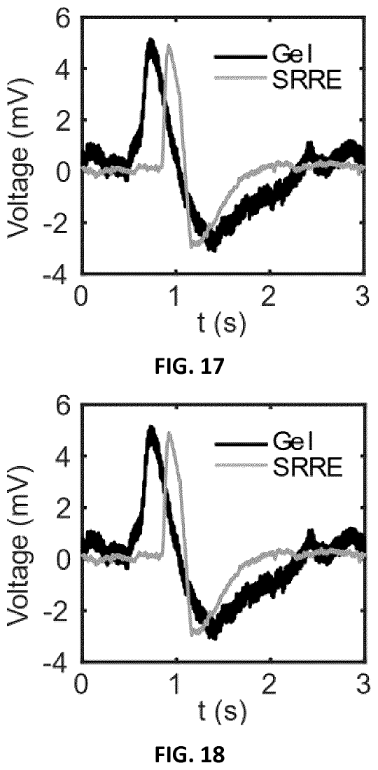

The SRRE was also tested as a reference electrode for an electrocardiogram (FIG. 16) or an electroencephalogram (FIGS. 17-18). FIG. 16 shows the measured voltage from lead 1 electrocardiogram signal across the forearm of an individual. By comparing the measured voltage using the disposable gel electrode to the SRRE, it was found that there is significantly less noise in the data when the SRRE is used as the bioelectrode. FIG. 17 shows the measured the voltage of the electroencephalogram signal from the forehand to the back of the head of an individual. Similarly to the electrocardiogram, a significant decrease in noise was observed compared to the gel electrode, and thus a higher signal-to-noise ratio is achieved when the SRRE is used instead of the traditional disposable gel electrode. FIG. 18 shows the background noise of the measured electroencephalogram voltage signal. The direct comparison of the background noise shows a reduction of noise power by 50 percent, when the SRRE is used instead of the traditional disposable gel electrode.

While the disclosure has been described with particular reference to the illustrated embodiment, it will be understood that numerous modifications thereto will appear to those skilled in the art. Accordingly, the above description and accompanying drawings should be taken as illustrative and not in a limiting sense. The arrows 111 and 113 of FIG. 1 are only schematic representations of the first and second channels, respectively. These schematic representations are not meant to be limitative in any way. For instance, the first and second channels are not necessarily linear or vertical as they can have any arbitrary form, shape, size and orientation, depending on the embodiment. While the disclosure has been described in connection with specific embodiments thereof, it will be understood that it is capable of further modifications and this application is intended to cover any variations, uses, or adaptations and including such departures as come within known or customary practice within the art and as may be applied to the essential features hereinbefore set forth, and as follows in the scope of the appended claims.

1. A solid reservoir reference electrode comprising:
a first layer of a given metal;
a second layer of a salt of the given metal and a non-metallic species atop the first layer;
a third layer of an electrolyte salt atop the second layer, the electrolyte salt including the non-metallic species; and
a fourth layer of an inert polymer atop the third layer;
wherein the electrolyte salt is soluble in an electrolytic solution receivable atop the fourth layer; the fourth layer, when in contact with the electrolyte solution, has first channels allowing the electrolytic solution to flow through the fourth layer; the third layer has second channels formed therein upon dissolution of the electrolyte salt by the electrolytic solution flowing through the first channels of the fourth layer; and the electrolytic solution is in fluid communication with the salt of the given metal of the second layer through the first channels and the second channels.

2. The solid reservoir reference electrode of claim 1, wherein the inert polymer is porous and has the first channels incorporated therein.

3. The solid reservoir reference electrode of claim 1, wherein the inert polymer has a sacrificial material incorporated therein, the sacrificial material is soluble in the electrolytic solution, wherein when the inert polymer comes into contact with the electrolytic solution, the first channels are formed therein upon dissolution of the sacrificial material.

4. The solid reservoir reference electrode of claim 1, wherein the third layer includes a crystalline, a polycrystalline, and/or a powdered form of the electrolyte salt.

5. The solid reservoir reference electrode of claim 1, wherein the electrolyte salt has a grain size of less than 20 μm.

6. The solid reservoir reference electrode of claim 1, wherein the given metal is selected from Ag, Cu, Zn, Au, Pt, Al, Cr, Ni, Sn, Fe, Co, oxides thereof and doped alloys thereof.

7. The solid reservoir reference electrode of claim 1, wherein the non-metallic species is selected from Cl⁻, Br⁻, F⁻, I⁻, SO₄²⁻, S²⁻, OH⁻, CO₃²⁻, PO₄³⁻, HCO₃⁻, HPO₄², H₂PO₄⁻, PF₆⁻, BF₄⁻, NO₃⁻, a formate anion, an acetate anion, an oxalate anion, a citrate anion, ethylenediaminetetraacetic acid (EDTA), a sulfonate anion, dicyanamide, bistriflimide, a cyclopentadienyl anion and derivatives thereof, and a pyridine anion and derivatives thereof.

8. The solid reservoir reference electrode of claim 1, wherein the electrolyte salt is selected from NaCl, Na₂SO₄, (NH₄)Cl, LiCl, RbCl, CaCl₂, KCl, MgCl₂, tetraethylammonium chloride (TEACl), tetrabutylammonium chloride (TBACl), K₂SO₄, Na₂S, or K₂S.

9. The solid reservoir reference electrode of claim 1, wherein the electrolyte salt is a halogen containing salt and the salt of the given metal is a salt of the given metal and the halogen.

10. The solid reservoir reference electrode of claim 9, wherein the electrolyte salt is KCl, NaCl, CaCl₂, or MgCl₂.

11. The solid reservoir reference electrode of claim 1, wherein the salt of the given metal is Ag₂S, CuS, CuSO₄, Ag₂SO₄, CuCl₂, AgCl, NiS, NiSO₄, NiCl₂, AuS, Au₂SO₄, AuCl, PtS, PtSO₄ or PtCl₂.

12. The solid reservoir reference electrode of claim 11, wherein the electrolyte salt is K₂S, Na₂S, Na₂SO₄ or K₂SO₄.

13. The solid reservoir reference electrode of claim 1, wherein a mass ratio of the inert polymer to the sacrificial material incorporated therein is from 0.1 to 1.

14. (canceled)

15. The solid reservoir reference electrode of claim 1, wherein the electrolytic solution is an aqueous solution and the sacrificial material incorporated in the inert polymer is hygroscopic.

16. The solid reservoir reference electrode of claim 1, wherein the sacrificial material is a saccharide, a salt or the electrolyte salt.

17. The solid reservoir reference electrode of claim 1, further comprising a substrate layer supporting the first layer.

18. (canceled)

19. A potentiometric sensor comprising the reference electrode of claim 1, and at least one of a working electrode and an ion sensitive field effect transistor, the potentiometric sensor being one of: a biosensor, a wrist strap, and a lest strip.

20. (canceled)

21. (canceled)

22. An array of potentiometric sensors comprising the reference electrode of claim 1 and at least one of an array of working electrodes and ion sensitive field effect transistors.

23. A solid reservoir reference electrode comprising:
a first layer of a given metal;
a second layer of a salt of the given metal and a non-metallic species atop the first layer;
a third layer of an electrolyte salt atop the second layer, the electrolyte salt including the non-metallic species; and
a fourth layer of an inert polymer atop the third layer.

24. (canceled)

25. The solid reservoir reference electrode of claim 23 wherein the inert polymer is porous.

26. (canceled)
